(19) United States
(12) Patent Application Publication
(10) Pub. No.: US 2018/0192885 A1
(43) Pub. Date: Jul. 12, 2018

(54) AUXILLIARY THERMOMETER AND THERMAL DETECTING METHOD THEREOF

(71) Applicant: RADIANT INNOVATION INC., HSINCHU CITY (TW)

(72) Inventors: NIEN-YU WENG, HSINCHU CITY (TW); HSIAO-YU TSAI, HSINCHU CITY (TW)

(21) Appl. No.: 15/403,953

(22) Filed: Jan. 11, 2017

(51) Int. Cl. A61B 5/01; G01K 1/14; G01K 13/00; G01K 7/22; G01K 3/08; A61B 5/00

(52) U.S. Cl. CPC A61B 5/01; G01K 1/14; G01K 13/002; A61B 5/6831; G01K 3/08; A61B 5/6824; A61B 5/6823; G01K 7/22

(57) ABSTRACT

This instant disclosure provides an auxiliary thermometer being adapted for measuring a human body temperature between an arm and a trunk of a human body including a carrying module, a temperature measurement module, a control module and a fixed structure. The temperature measurement module includes a first temperature measurement unit disposed on the carrying module and a second temperature measurement unit opposite to the first temperature measurement unit and correspondingly disposed on the carrying module. The control module is disposed on the carrying module and electrically connected to the first temperature measurement unit and the second temperature measurement unit. The carrying module is disposed on the fixed structure to be disposed on the arm or the trunk of the human body.

### BACKGROUND OF THE INVENTION

### 1. Field of the Invention

The instant disclosure relates to a thermometer and a thermal detecting method thereof; in particular, to an auxiliary thermometer being adapted for measuring an auxiliary temperature and a thermal detecting method thereof.

### 2. Description of Related Art

There are many kinds of thermometers in the market, most are electrical thermometers or infrared ear thermometers etc., and are single-use thermometers for instant temperature measurement. However, when the user conducts an instant temperature measurement using the present auxiliary thermometers, the user has to tightly hold the auxiliary thermometer with his/her arm for a while to steady the auxiliary temperature to obtain an accurate temperature information.

Although there is a wearable auxiliary thermometer developed to be worn on the user for long-term monitoring, the wearable auxiliary thermometer no matter whether it is an attachable thermometer being attached to the trunk of a human body or it is a thermometer being strapped to fasten onto the user's body with a belt, has only one temperature sensor being positioned at the armpit to conduct the auxiliary temperature measurement.

Therefore, when the user uses the aforementioned wearable auxiliary thermometer, to obtain an accurate temperature information, the user has to tightly hold the auxiliary thermometer with his/her arm for a long time to obtain a steady and accurate auxiliary temperature. However, in actual practice, the user's arm still moves, such that a tightness between the arm and the trunk would be different, causing the measured auxiliary temperature to have a fluctuation, and the magnitude of the fluctuation is influenced by the difference of the tightness between the arm and the trunk and by the thermometer when the arm opens and the environmental temperature at that time influences it. If the magnitude of the fluctuation of the auxiliary temperature which is caused by the user opening his/her arm or by the change of the actual temperature value (fever or bring down a fever) cannot be distinguished, the long-term monitoring has less effect.

Therefore, how to provide an auxiliary thermometer and a thermal detecting method thereof which is available for long-term monitoring of the user's body temperature to solve the aforementioned problem are important issues in the art.

### SUMMARY OF THE INVENTION

In order to overcome the abovementioned problem, this instant disclosure provides an auxiliary thermometer and a thermal detecting method to compensate temperature differences using first and second temperature measurement units so as to upgrade an accuracy of measuring temperature and achieve long-term monitoring.

To achieve the abovementioned purpose, one of the embodiments of this instant disclosure provides an auxiliary thermometer which is adapted for measuring a human body temperature between an arm and a trunk of a human body, and the auxiliary thermometer includes a carrying module, a temperature measurement module, a control module, and a fixed structure. The temperature measurement module includes a first temperature measurement unit disposed on the carrying module and a second temperature measurement unit opposite to the first temperature measurement unit and correspondingly disposed on the carrying module. The control module is disposed on the carrying module and electrically connected to the first temperature measurement unit and the second temperature measurement unit. The carrying module is disposed on the fixed structure to be disposed on the arm or the trunk of the human body.

Another embodiment of this instant disclosure provides a thermal detecting method of an auxiliary thermometer which is adapted for measuring a human body temperature between an arm and a trunk of a human body, and the thermal detecting method of the auxiliary thermometer includes the following steps. A temperature measurement module being electrically connected to a control module and including a first temperature measurement unit and a second temperature measurement unit is provided. The temperature of the arm or the trunk is measured by the first temperature measurement unit to obtain a first temperature measurement information. A space between the arm and the trunk is measured by the second temperature measurement unit to obtain a second temperature measurement information. The control module obtains a corresponding auxiliary temperature measurement information according to the first temperature measurement information and the second temperature measurement information.

A yet another embodiment of this instant disclosure provides a thermal detecting method of an auxiliary thermometer which is adapted for measuring a human body temperature between an arm and a trunk of a human body, and the thermal detecting method of the auxiliary thermometer includes the following steps. A temperature measurement module being electrically connected to a control module and including a first temperature measurement unit and a second temperature measurement unit is provided. The temperature of the arm or the trunk is measured by the first temperature measurement unit to obtain a first temperature measurement information. A space between the arm and the trunk is measured by the second temperature measurement unit to obtain a second temperature measurement information. A difference between the first temperature measurement information and the second temperature measurement information is calculated. Whether the difference is bigger than a predetermined temperature difference threshold is determined, when the difference is bigger than the predetermined temperature difference threshold, the control module records or sends a prompting message.

This instant disclosure has the benefit that, the auxiliary thermometer and the thermal detecting method thereof of this instant disclosure has the technical feature of “a first temperature measurement unit disposed on the carrying module and a second temperature measurement unit opposite to the first temperature measurement unit and correspondingly disposed on the carrying module” that can perform the effect of compensating temperature differences so as to upgrade an accuracy of measuring temperature and achieve long-term monitoring.

In order to further appreciate the characteristics and technical contents of the present invention, references are hereunder made to the detailed descriptions and appended drawings in connection with the instant disclosure. However, the appended drawings are merely shown for exemplary purposes, rather than being used to restrict the scope of the instant disclosure.

### BRIEF DESCRIPTION OF THE DRAWINGS

FIG. 1 shows a module block diagram of an auxiliary thermometer of an embodiment in the instant disclosure;

FIG. 2 shows a perspective schematic view of an auxiliary thermometer of an embodiment in the instant disclosure;

FIG. 3 is one of the fastening methods of an auxiliary thermometer of an embodiment in the instant disclosure;

FIG. 4 is another fastening method of an auxiliary thermometer of an embodiment in the instant disclosure;

FIG. 5 shows a flowchart of a thermal detecting method of an auxiliary thermometer of an embodiment in the instant disclosure;

FIG. 6 is one of the states of use of an auxiliary thermometer of an embodiment in the instant disclosure;

FIG. 7 is another state of use of an auxiliary thermometer of an embodiment in the instant disclosure;

FIG. 8 is yet another state of use of an auxiliary thermometer of an embodiment in the instant disclosure; and

FIG. 9 shows a representative figure demonstrating a change in human body temperature measured by an auxiliary thermometer of an embodiment in the instant disclosure.

### DETAILED DESCRIPTION OF THE PREFERRED EMBODIMENTS

Embodiments of an auxiliary thermometer and a thermal detecting method disclosed in the instant disclosure are illustrated via specific examples as follows. People familiar in the art may easily understand the advantages and efficacies of the instant disclosure by disclosure of the specification. The instant disclosure may be implemented or applied by other different specific examples, and each of the details in the specification may be applied based on different views and may be modified and changed under the existence of the spirit of the instant disclosure. The figures in the instant disclosure are only for brief description, but they are not depicted according to actual size and do not reflect the actual size of the relevant structure. The following embodiments further illustrate related technologies of the instant disclosure in detail, but the scope of the instant disclosure is not limited herein.

In this specification, the terminology such as first, second, or third is used for describing various elements or information, but the elements or information should not be restricted by these terminologies. These terminologies are used for distinguishing between an element and another element, or a piece of information and another piece of information. In addition, the terminology used in this specification can include any one of or a plurality of combinations of related items depending upon the situation.

### First Embodiment

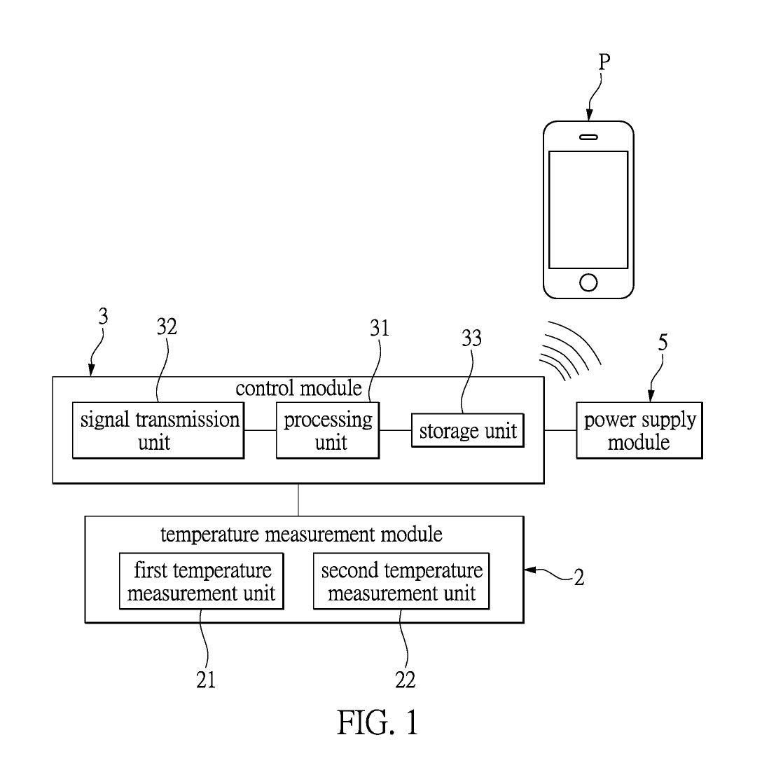

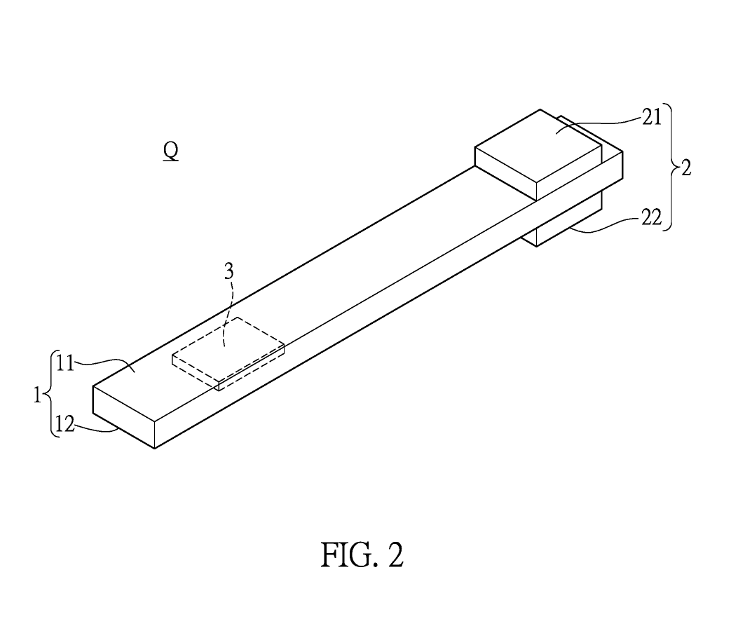

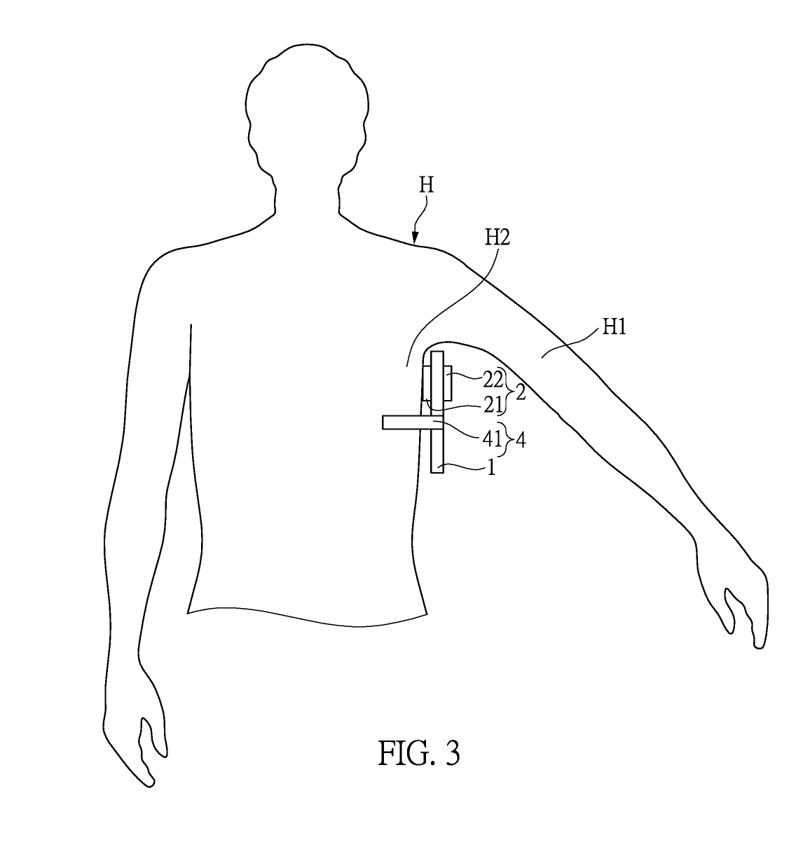

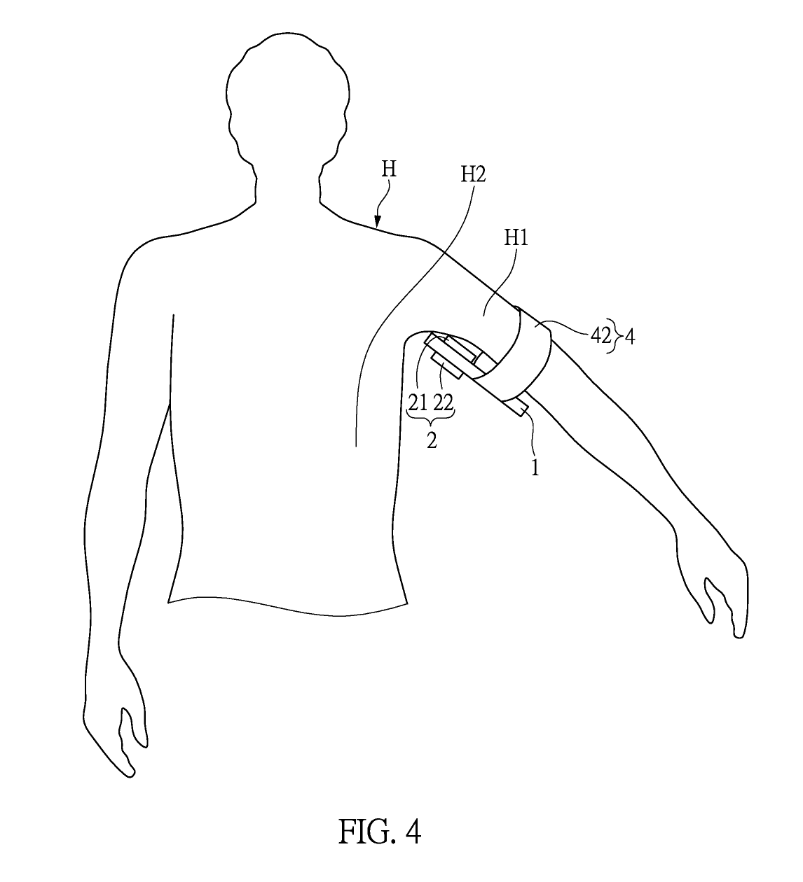

Firstly, please refer to FIG. 1 and FIG. 2, and coordinate with FIG. 3 and FIG. 4. The first embodiment of this instant disclosure provides an auxiliary thermometer Q which is adapted for measuring a human body temperature between an arm H1 and a trunk H2 of a human body H, and the auxiliary thermometer Q includes a carrying module 1, a temperature measurement module 2, a control module 3, and a fixed structure 4. For instance, the human body temperature (e.g., auxiliary temperature) measured by the temperature measurement module 2 can be transmitted to an electronic device P via a signal transmission function of the control module 3, and a long-term monitoring of a user's temperature is conducted.

Next, as shown in FIG. 1 and FIG. 2, in this embodiment of the instant disclosure, the temperature measurement module 2 includes a first temperature measurement unit 21 disposed on the carrying module 1 and a second temperature measurement unit 22 opposite to the first temperature measurement unit 21 and correspondingly disposed on the carrying module 1. For instance, the first temperature measurement unit 21 and the second temperature measurement unit 22 may be a thermistor thermometer, but it is not limited herein. Preferably, the first temperature measurement unit 21 and the second temperature measurement unit 22 are a sheet-shaped temperature sensor to be disposed on the carrying module 1.

The carrying module 1 has a first surface 11 (upper surface) and a second surface 12 (lower surface) relative to the first surface 11. Preferably, in this embodiment of the instant disclosure, the first surface 11 and the second surface 12 are external surfaces that are opposite to each other. However, in other embodiments, the first surface 11 and the second surface 12 may also be adjacently disposed. In such a way, the first temperature measurement unit 21 can be disposed on the first surface 11 of the carrying module 1, and the second temperature measurement unit 22 can be disposed on the second surface 12 of the carrying module 1. In other words, there are at least two temperature measurement units (first temperature measurement unit 21 and second temperature measurement unit 22) disposed on the carrying module to independently measure temperature. The carrying module 1 may be a substrate, for example, the substrate can be a printed circuit board (PCB), but it is not limited in this instant disclosure.

The control module 3 may be disposed on the carrying module 1, and may be electrically connected to the temperature measurement module 2, that is the control module 3 may be electrically connected to the first temperature measurement unit 21 and second temperature measurement unit 22. In addition, for instance, the control module 3 may be electrically connected to the carrying module 1 and pass through the carrying module 1 to electrically connect to the temperature measurement module 2.

In this embodiment of this instant disclosure, the control module 3 may be a microcontroller unit (MCU), but it is not limited in this instant disclosure. In other embodiments, the control module 3 may be an application-specific integrated circuit (ASIC). Moreover, the control module 3 further includes a processing unit 31, a signal transmission unit 32 and a storage unit 33. The processing unit 31 may be used for integrating a first temperature measurement information T1 (a temperature value obtained by the first temperature measurement unit 21) and a second temperature measurement information T2 (a temperature value obtained by the second temperature measurement unit 22) measured by the first temperature measurement unit 21 and the second temperature measurement unit 22 independently, so as to obtain an auxiliary temperature measurement information T. Furthermore, in order to conduct long-term monitoring on a user's human body temperature, the storage unit 33 may be used to store the user's auxiliary temperature measurement information T at different time points. Additionally, the signal transmission unit 32 may be used to instantly or intermittently transmit the user's auxiliary temperature measurement information T to an electronic device P by wired or wireless transmission means. For example, the electronic device P can be a smartphone or a tablet, and the signal transmission unit 32 can be a cable or a blue tooth, but the transmission means is not limited to a signal transmission means in this instant disclosure. Thus, the auxiliary thermometer Q may have a connection port to connect to the exterior electronic device P, or may supply a power to the auxiliary thermometer Q via the connection port.

Additionally, the auxiliary thermometer Q may further include a power supply module 5 which may electrically connect to and supply the power to the control module 3 and the temperature measurement module 2. For instance, for adapting to the portable auxiliary thermometer Q, the power supply module 5 can be a battery or other power sources which can store electric energy. However, in other embodiments, the power supply module 5 may be the power supply provided from a socket, and the auxiliary thermometer Q can thus supply the power via a power supply cord (not shown).

Next, please refer to FIG. 3 and FIG. 4. The carrying module 1 disposed with the temperature measurement module 2 and the control module 3 may be disposed on the fixed structure 4 to be set on the arm H1 or the trunk H2 of the human body H. For example, the fixed structure 4 can be various types such as the embodiment of FIG. 3, the fixed structure 4 can be an adhesive colloid 41 which can be an adhesive tape, a two-sided adhesive tape, or other adhesives for adhering the carrying module 1 to be set on the arm H1 or the trunk H2 of the human body H. The carrying module 1 can thus be disposed on the arm H1 or the trunk H2 through the adhesive colloid 41. Moreover, in the embodiment of FIG. 4, the fixed structure 4 may be a belt 42 which can be a binding band, and the carrying module 1 can thus surround the user's trunk H2 or arm H1 via the belt 42 to set the auxiliary thermometer Q on the arm H1 or the trunk H2, so as to conduct the human body temperature measurement.

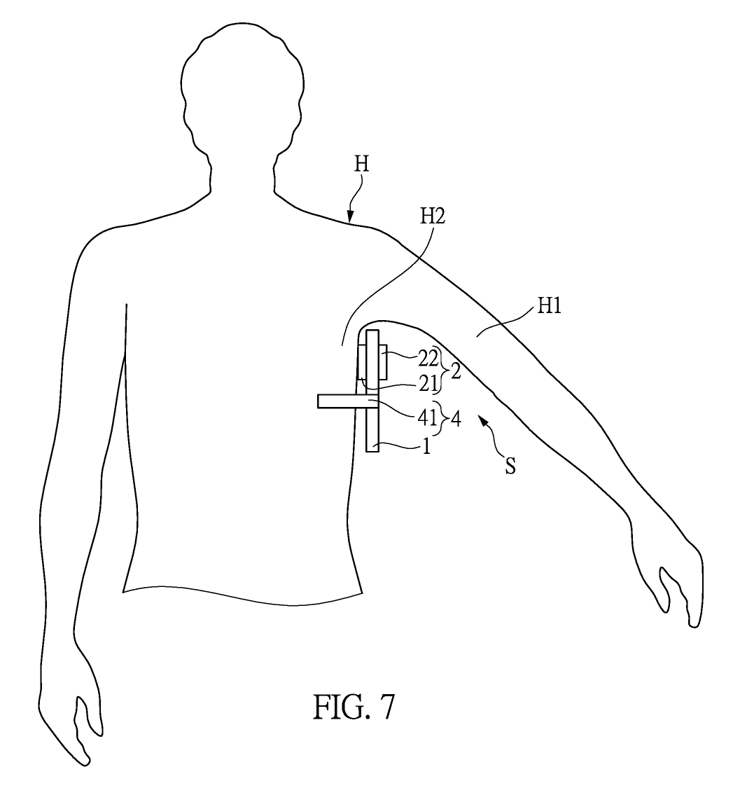

Referring to FIG. 3 and FIG. 4 again, specifically, as shown in FIG. 3, the carrying module 1 may be disposed on the trunk H2 of the human body H through the fixed structure 4 to tightly bind the first temperature measurement unit 21 to the skin of the trunk H2. At this time, when the first temperature measurement unit 21 is fixed to and contacted with the trunk H2 by the fixed structure 4, the second temperature measurement unit 22 can be tightly held by the arm H1 to contact with the arm H1, or can be exposed to the space S (referring to FIG. 7) formed between an inner side of the arm H1 and the trunk H2 by the arm H1 opening.

Furthermore, as shown in FIG. 4, the carrying module 1 may be disposed on the arm H1 of the human body H through the fixed structure 4 to tightly bind the first temperature measurement unit 21 to the skin of the arm H1. At this time, when the first temperature measurement unit 21 is fixed to and contacted with the arm H1 by the fixed structure 4, the second temperature measurement unit 22 can be tightly held by the arm H1 to contact with the trunk H2, or can be exposed to the space S formed between the inner side of the arm H1 and the trunk H2 by the arm H1 opening. Since the auxiliary thermometer Q of this instant disclosure can be disposed on the arm H1 or the trunk H2 through the fixed structure 4, the user can wear the auxiliary thermometer Q of this instant disclosure with clothes on. Therefore, the second temperature measurement unit 22 measures the space S between the inner side of the arm H1 and the trunk H2 covered by the clothes. In other embodiments, a sheet of the colloid may be directly used to completely wrap the first temperature measurement unit 21 and the second temperature measurement unit 22 to bind to the arm H1 or the trunk H2.

By this way of disposing the at least two temperature measurement units (first temperature measurement unit 21 and second temperature measurement unit 22), the temperature measurement information measured by the two temperature measurement units (first temperature measurement unit 21 and second temperature measurement unit 22) can be compensated with each other to obtain the auxiliary temperature measurement information T, and methods of the temperature compensation are described in a second embodiment.

### Second Embodiment

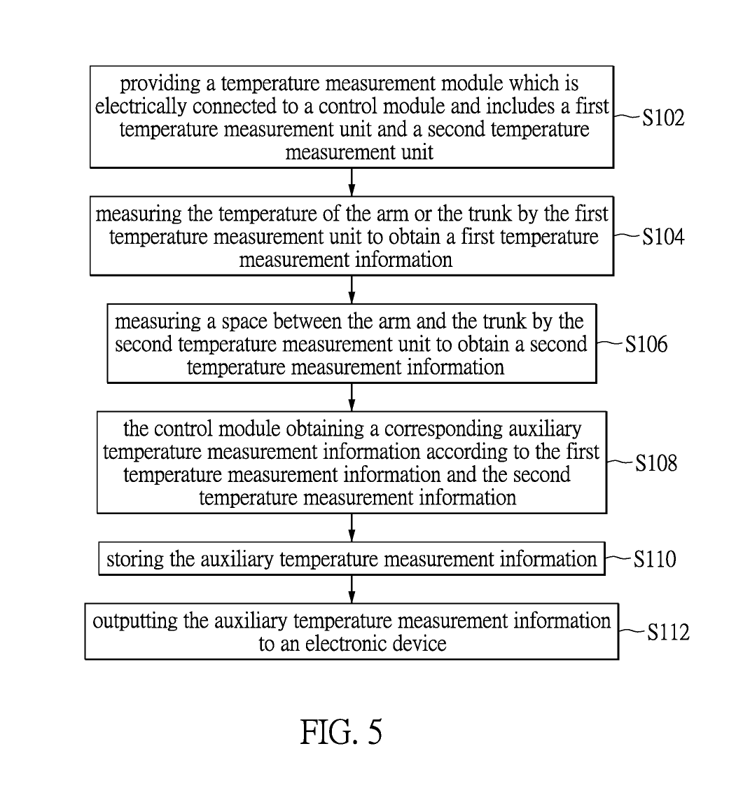

Firstly, please refer to FIG. 5, and coordinate with FIG. 1 and FIG. 2. The second embodiment of this instant disclosure provides a thermal detecting method of an auxiliary thermometer Q. In the second embodiment, the embodiment of FIG. 3 is illustrated for explanation, that is, the carrying module 1 is disposed on the arm H1 of the human body H through the fixed structure 4 to tightly bind the first temperature measurement unit 21 to the skin of the trunk H2, and the second temperature measurement unit 22 can be tightly held by the arm H1 to contact with the arm H1, or can be exposed to the space S formed between the arm H1 and the trunk H2 by the arm H1 opening. However, in other embodiments, the embodiment of FIG. 4 may be used to conduct the human body temperature measurement, but it is not limited in this instant disclosure.

Specifically, please refer to FIG. 5 again, the thermal detecting method of the auxiliary thermometer Q of this instant disclosure is adapted for measuring a human body temperature between an arm H1 and a trunk H2 of a human body H, which includes the following steps. As shown in step S102, a temperature measurement module 2 is provided, and is electrically connected to a control module 3. The temperature measurement module 2 includes a first temperature measurement unit 21 and a second temperature measurement unit 22. In detailed description, the specific temperature measurement module 2 is identical to that of the first embodiment, the temperature measurement module 2 may include a first temperature measurement unit 21 and a second temperature measurement unit 22 that are disposed on a carrying module 1, so that the first temperature measurement unit 21 can contact with the trunk H2 of the human body H, and the second temperature measurement unit 22 can contact with or detach from the arm H1 by a movement of the arm H1. Other structures of the auxiliary thermometer Q are similar to that of the aforementioned first embodiment, thus they are not repeated herein.

Next, as shown in step S104, the temperature of the arm H1 or the trunk H2 is measured by the first temperature measurement unit 21 to obtain a first temperature measurement information T1. Specifically, in the second embodiment, since the implementation method of FIG. 3 is illustrated for explanation, the first temperature measurement unit 21 can thus obtain the temperature of the trunk H2. However, in other embodiments, the auxiliary thermometer Q may be disposed as shown in FIG. 4 to conduct the human body temperature measurement. In other words, via the implementation method of FIG. 4, the first temperature measurement unit 21 can constantly contact with the skin of the inner side of the arm H1.

Next, as shown in step S106, a space S between the inner side of the arm H1 and the trunk H2 is used by the second temperature measurement unit 22 to obtain a second temperature measurement information T2. Specifically, since the user's inner side of the arm H1 cannot tightly contact with the trunk H2 for a long time, the arm H1 would thus open to move away from the trunk H2 in some situations, such that a space S is formed between the inner side of the arm H1 and the trunk H2. Furthermore, the second temperature measurement unit 22 is used to measure the second temperature measurement information T2 of the space S located between the inner side of the arm H1 and the trunk H2.

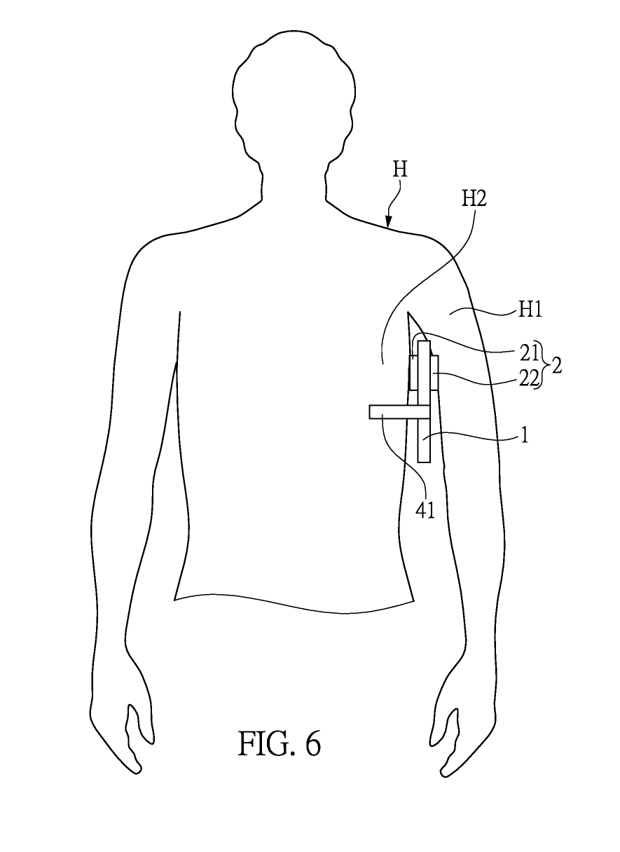

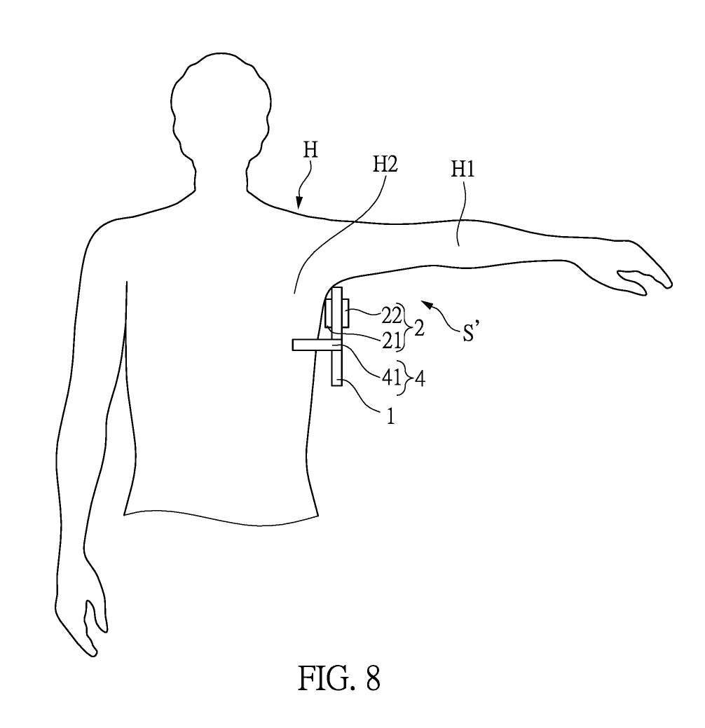

Then, firstly, please refer to FIG. 6 and FIG. 8, which describe a swing amplitude of the user's arm H1 affecting the first temperature measurement information T1 and the second temperature measurement information T2 measured by the first temperature measurement unit 21 and the second temperature measurement unit 22 respectively. In detailed description, FIG. 6 shows the inner side of the user's arm H1 facing toward and tightly contacting with the trunk H2. It should be noted that, in order to clearly show the first temperature measurement unit 21 and the second temperature measurement unit 22, the arm H1 represented in FIG. 6 does not tightly contact with the trunk H2, but in actual practice, the user's arm H1 can tightly contact with the trunk H2 to measure an accurate temperature value. In this way, as the method of use in FIG. 6, the first temperature measurement information T1 (temperature value) measured by the first temperature measurement unit 21 would be close to the second temperature measurement information T2 (temperature value) measured by the second temperature measurement unit 22.

Continuing from the above, please refer to FIG. 7, when the user is moving, a space S would be formed between the inner side of the arm H1 and the trunk H2, and if influences of the first temperature measurement information T1 and the second temperature measurement information T2 measured by the first temperature measurement unit 21 and the second temperature measurement unit 22 respectively are not too much, the human body temperature still can be obtained by the method of the temperature compensation of the first temperature measurement information T1 and the second temperature measurement information T2. In other words, because the human body temperature is generally higher than the surrounding temperature, hence, when the arm H1 slightly opens, the temperature values obtained by the first temperature measurement information T1 and the second temperature measurement information T2 would decrease. If the temperature difference obtained by the first temperature measurement information T1 and the second temperature measurement information T2 is small, the method of the temperature compensation can be used to obtain the human body temperature. It should be noted that, since the second temperature measurement unit 22 is disposed at the space S formed between the inner side of the arm H1 and the trunk H2, the influence caused by external environmental factors of second temperature measurement information T2 would be more severe than that of the first temperature measurement information T1. Generally speaking, when an external temperature is lower than the human body temperature, the temperature value of the second temperature measurement information T2 is lower than that of the first temperature measurement information T1.

Continuing from the above, please refer to FIG. 8, when the user's arm H1 opens substantially, a space S′ formed between the inner side of the arm H1 and the trunk H2 would significantly influence the first temperature measurement information T1 and the second temperature measurement information T2 measured by the first temperature measurement unit 21 and the second temperature measurement unit 22 respectively. Therefore, the temperature difference obtained by the first temperature measurement information T1 and the second temperature measurement information T2 is too big to obtain the human body temperature of the method of the temperature compensation.

Then, please refer to FIG. 5 again, as shown in step S108, the control module 3 obtains a corresponding auxiliary temperature measurement information T according to the first temperature measurement information T1 and the second temperature measurement information T2. Specifically, the first temperature measurement information T1 and the second temperature measurement information T2 may be transmitted into the control module 3 to proceed with the temperature compensation to obtain the auxiliary temperature measurement information T. For example, the control module 3 can calculate the auxiliary temperature measurement information T according to a temperature calculation formula which meets a following correlation: T=T1+K*(T1-T2), wherein T is the auxiliary temperature measurement information, T1 is the first temperature measurement information, T2 is the second temperature measurement information, and K is a compensation coefficient. The unit of Celsius temperature is used in this instant disclosure, however, in other embodiments, other temperature units may be used for calculation.

In the step S108, during the step of obtaining the auxiliary temperature measurement information T, preferably further including calculating a difference D between the first temperature measurement information T1 and the second temperature measurement information T2. More in detail, the difference D can be obtained by subtracting the first temperature measurement information T1 from the second temperature measurement information T2. Under different conditions, the difference D can correspond to different compensation coefficients K (such as first compensation coefficient K1 or second compensation coefficient K2) at different difference intervals. In other words, a plurality of difference intervals can correspond to a plurality of compensation coefficients respectively, the plurality of difference intervals can be continuous intervals, and each of the intervals has its own corresponding compensation coefficient. Hereinafter, two difference intervals (such as a first difference interval and second difference interval) are described. However, in other embodiments, it is also possible to set more of a plurality of difference intervals (such as third difference interval and fourth difference interval) to correspond different values of differences.

Continuing from the above, in detail, after obtaining the difference D between the first temperature measurement information T1 and the second temperature measurement information T2, during the step of obtaining the auxiliary temperature measurement information T, the step S108 may further include determining the difference D being positioned in one of a plurality of difference intervals, the plurality of difference intervals correspond with the plurality of compensation coefficients respectively, so as to respectively correspondingly be brought into the plurality of compensation coefficients to calculate the auxiliary temperature measurement information T. For instance, a first difference interval and a second difference interval can be included in the plurality of difference intervals, and a first compensation coefficient K1 and a second compensation coefficient K2 can be included in the plurality of compensation coefficients K, such that the difference D can be determined being positioned in a first difference interval or in a second difference interval. When the difference D is positioned in the first difference interval, a first compensation coefficient K1 is brought into the compensation coefficients K of the temperature calculation formula to calculate. In addition, when the difference D is positioned in the second difference interval, a second compensation coefficient K2 is brought into the compensation coefficients K of the temperature calculation formula to calculate.

Then, for example, when the difference D between the first temperature measurement information T1 and the second temperature measurement information T2 (that is T1-T2=D) is in the first difference interval (that is 0≤D<0.3□), the first compensation coefficient K1 can be brought into the temperature calculation formula. When the difference D between the first temperature measurement information T1 and the second temperature measurement information T2 is in the second difference interval (that is 0.3≤D<0.6□), the second compensation coefficient K2 can be brought into the temperature calculation formula. When the difference D between the first temperature measurement information T1 and the second temperature measurement information T2 is in the third difference interval R3 (that is 0.6≤D<0.9□), the third compensation coefficient K3 can be brought into the temperature calculation formula. In other embodiments, the compensation coefficients K may be a diagonal equation or a curve equation, and the compensation coefficients K may be changed according to various differences D.

Continuing, please refer to FIG. 5 and FIG. 8, the step S108, the difference D between the first temperature measurement information T1 and the second temperature measurement information T2 being obtained during the step of obtaining the auxiliary temperature measurement information T, may further include determining whether the difference D is bigger than a predetermined temperature difference threshold, and when the difference D is bigger than the predetermined temperature difference threshold, the control module 3 can record, store or send a prompting message. Specifically, when the arm H1 opens substantially, the difference D between the first temperature measurement information T1 and the second temperature measurement information T2 might be too big, thus when the difference D is bigger than the predetermined temperature difference threshold, a prompting message can be sent to the electronic device P to remind a monitor, and to suggest the monitor to not involve these reading values for reference. That is, the control module 3 can store the difference D measured during the period of time first, and, at the same time, can record the auxiliary temperature measurement information T calculated during the period of time to determine whether it has availability.

Then, please refer to FIG. 5 again, as shown in step S110, the auxiliary temperature measurement information T is stored. For example, in order to monitor a user's human body temperature for a long time, the auxiliary thermometer Q has to be worn for long time, thus, when obtaining each of the pieces of auxiliary temperature measurement information T, the obtained auxiliary temperature measurement information T can be stored in the storage unit 33 of the control module 3. When the difference D is bigger than the predetermined temperature difference threshold, not only can a prompting message be sent to the electronic device P, but also the calculated auxiliary temperature measurement information T can be stored at the same time. However, in this instant disclosure, it is not limited to whether it stores the auxiliary temperature measurement information T under the condition of the difference D being bigger than the predetermined temperature difference threshold.

Next, as shown in step S112, the auxiliary temperature measurement information T is outputted to an electronic device P. Specifically, the electronic device P of the second embodiment is similar to the electronic device P of the aforementioned first embodiment. By this way, when the auxiliary thermometer Q is worn on an infant or a toddler, the signal transmission unit 32 of the control module 3 can be used to transmit a plurality of auxiliary temperature measurement information T to his/her parent's or monitor's mobile phone by wired or wireless transmission means, so as to understand the change of the infant's/toddler's body temperature over a long time.

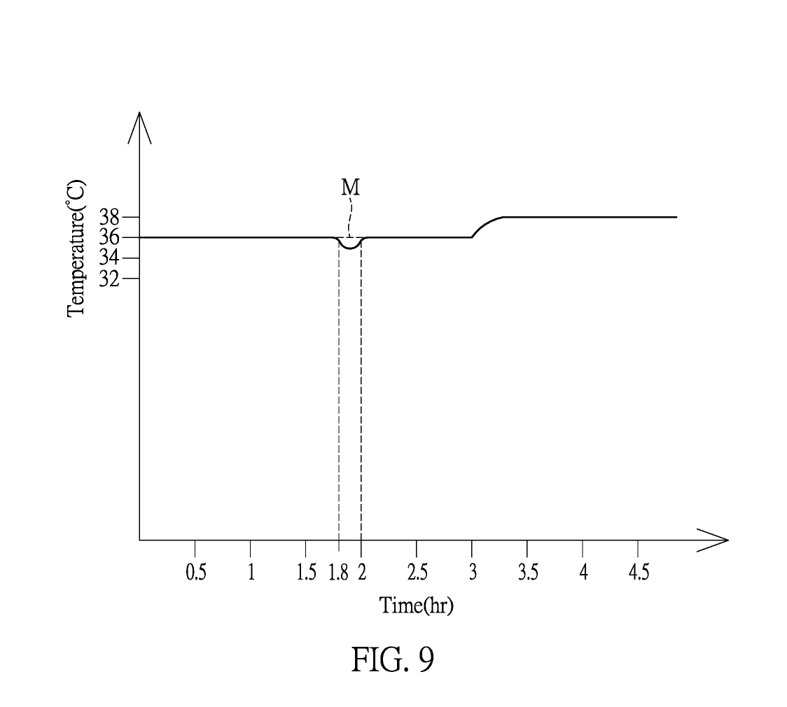

Please refer to FIG. 9. FIG. 9 shows the change of the user's body temperature over a long period of time (such as 4.5 hours shown in FIG. 9). It can be read from FIG. 9, the user's body temperature has decreased between 1.8 hours and 2 hours, the transient change in body temperature means that the user's arm H1 may have opened slightly resulting in a temperature drop. Thus, the aforementioned thermal detecting method of the auxiliary thermometer Q can be used to compensate that temperature to generate a compensation temperature M.

### Third Embodiment

Please refer to FIG. 1, FIG. 4 and FIG. 5. The third embodiment of this instant disclosure provides a thermal detecting method of an auxiliary thermometer Q. In the third embodiment, the embodiment of FIG. 4 is illustrated for explanation, that is, the carrying module 1 is disposed on the arm H1 of the human body H through the fixed structure 4 to tightly bind the first temperature measurement unit 21 to the arm H1, and the second temperature measurement unit 22 can be tightly held by the arm H1 to contact with the trunk H2, or can be exposed to the space S formed between the arm H1 and the trunk H2 by the arm H1 opening. It should be noted that, since the temperature measurement module 2 is disposed on the arm H1 through the fixed structure 4, the amount of the compensation coefficient K would be influenced. Hereinafter, only the difference of the compensation coefficient K will be described.

Next, in detail, the control module 3 may calculate the auxiliary temperature measurement information T′ according to a temperature calculation formula which meets a following correlation: T′=T1′+K′*(T1′−T2′), wherein T′ is the auxiliary temperature measurement information, T1′ is the first temperature measurement information, T2′ is the second temperature measurement information, and K′ is a compensation coefficient. The difference D between the first temperature measurement information T1′ and the second temperature measurement information T2′ can be obtained by (T1 ‘−T2’). When the difference D is positioned in the first difference interval, a first compensation coefficient K1′ is brought into the compensation coefficient K′ of the temperature calculation formula to calculate. In addition, when the difference D is positioned in the second difference interval, a second compensation coefficient K2′ is brought into the compensation coefficients K′ of the temperature calculation formula for calculation.

In addition, the specific structures and proceeding steps of the thermal detecting method of the auxiliary thermometer Q in the third embodiment are similar to the aforementioned embodiments, thus it is not repeated herein.

### Benefits of Embodiments

In summary, this instant disclosure has the benefits that, the auxiliary thermometer Q and the thermal detecting method thereof of this instant disclosure has the technical feature of “a first temperature measurement unit 21 disposed on the carrying module 1 and a second temperature measurement unit 22 opposite to the first temperature measurement unit 21 and correspondingly disposed on the carrying module 1” that can perform the effect of compensating temperature differences so as to upgrade an accuracy of measuring temperature and achieve long-term monitoring.

Furthermore, via calculation of the temperature calculation formula and determination of the difference D, the present first temperature measurement information T1 and the second temperature measurement information T2 can be read and then it can be determined whether to proceed with the temperature compensation or not, and the compensation coefficient K obtained by actual experiments is brought into the temperature calculation formula to compensate the first temperature measurement information T1 and the second temperature measurement information T2 influenced by other external factors, so as to obtain the auxiliary temperature measurement information T.

The descriptions illustrated supra set forth simply the preferred embodiments of the present invention; however, the characteristics of the present invention are by no means restricted thereto. All changes, alterations, or modifications conveniently considered by those skilled in the art are deemed to be encompassed within the scope of the present invention delineated by the following claims.

What is claimed is:

1. An auxiliary thermometer being adapted for measuring a human body temperature between an arm and a trunk of a human body, comprising:
a carrying module;
a temperature measurement module including a first temperature measurement unit disposed on the carrying module and a second temperature measurement unit opposite to the first temperature measurement unit and correspondingly disposed on the carrying module;
a control module disposed on the carrying module and electrically connected to the first temperature measurement unit and the second temperature measurement unit; and
a fixed structure, the carrying module being disposed on the fixed structure to be disposed on the arm or the trunk of the human body.

2. The auxiliary thermometer as claimed in claim 1, wherein the fixed structure is an adhesive colloid, and the carrying module is disposed on the arm or the trunk by the adhesive colloid.

3. The auxiliary thermometer as claimed in claim 1, wherein the fixed structure is a belt, and the carrying module is disposed on the arm or the trunk by the belt.

4. The auxiliary thermometer as claimed in claim 1, wherein the first temperature measurement unit is constantly kept in contact with the arm or the trunk by the fixed structure.

5. A thermal detecting method of an auxiliary thermometer being adapted for measuring a human body temperature between an arm and a trunk of a human body, comprising the following steps:
providing a temperature measurement module which is electrically connected to a control module and includes a first temperature measurement unit and a second temperature measurement unit;
measuring the temperature of the arm or the trunk by the first temperature measurement unit to obtain a first temperature measurement information;
measuring a space between the arm and the trunk by the second temperature measurement unit to obtain a second temperature measurement information; and
the control module obtaining a corresponding auxiliary temperature measurement information according to the first temperature measurement information and the second temperature measurement information.

6. The thermal detecting method of the auxiliary thermometer as claimed in claim 5, wherein after the step of obtaining the auxiliary temperature measurement information, further comprises outputting the auxiliary temperature measurement information to an electronic device.

7. The thermal detecting method of the auxiliary thermometer as claimed in claim 5, wherein the control module calculates the auxiliary temperature measurement information according to a temperature calculation formula which meets a following correlation: T=T1+K*(T1-T2), wherein T is the auxiliary temperature measurement information, T1 is the first temperature measurement information, T2 is the second temperature measurement information, and K is a compensation coefficient.

8. The thermal detecting method of the auxiliary thermometer as claimed in claim 7, wherein during the step of obtaining the auxiliary temperature measurement information, further comprises calculating a difference between the first temperature measurement information and the second temperature measurement information.

9. The thermal detecting method of the auxiliary thermometer as claimed in claim 8, wherein during the step of obtaining the auxiliary temperature measurement information, further comprises determining whether the difference is bigger than a predetermined temperature difference threshold, when the difference is bigger than the predetermined temperature difference threshold, the control module records or sends a prompting message.

10. The thermal detecting method of the auxiliary thermometer as claimed in claim 8, wherein during the step of calculating the auxiliary temperature measurement information, further comprises determining whether the difference being positioned in one of a plurality of difference intervals, and the plurality of difference intervals correspond to and are brought into a plurality of compensation coefficients respectively to calculate the auxiliary temperature measurement information.

11. A thermal detecting method of an auxiliary thermometer being adapted for measuring a human body temperature between an arm and a trunk of a human body, comprising following steps:
providing a temperature measurement module which is electrically connected to a control module and includes a first temperature measurement unit and a second temperature measurement unit;
measuring the temperature of the arm or the trunk by the first temperature measurement unit to obtain a first temperature measurement information;
measuring a space between the arm and the trunk by the second temperature measurement unit to obtain a second temperature measurement information;
calculating a difference between the first temperature measurement information and the second temperature measurement information; and
determining whether the difference is bigger than a predetermined temperature difference threshold, when the difference is bigger than the predetermined temperature difference threshold, the control module records or sends a prompting message.
